(19) United States
(12) Patent Application Publication
(10) Pub. No.: US 2004/0132191 A1
(43) Pub. Date: Jul. 8, 2004

(54) Yeasts transformed by genes enhancing their cold stress tolerance

(76) Inventors: Emmanuelle Bourgeois, Montpellier (FR); Marie-Francoise Gautier, Montpellier (FR); Philippe Joudrier, Montpellier (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,145

(22) PCT Filed: Dec. 26, 2001
(86) PCT No.: PCT/FR01/04202

(30) Foreign Application Priority Data
Dec. 27, 2000 (FR) 00/17104

(51) Int. Cl. C12N 1/18; C12N 15/74; A21D 8/00

(52) U.S. Cl. 435/483; 435/254.2; 426/27

(57) ABSTRACT

The invention concerns yeasts whereof the cold stress tolerance is enhanced by transformation with at least one gene coding for a protein selected among a group 2 LEA protein, a WALI protein, or a LTP.

The invention relates to the production of yeasts resistant to stress, and in particular to cold stress.

Dough pieces which are inoculated beforehand with live yeasts and which generally undergo an onset of fermentation are used increasingly frequently in bread and pastry manufacture. They are then frozen and stored in this form until they are used. However, cold stress, caused by the freezing and preservation at negative temperatures, causes a considerable loss of yeast fermentative power in these dough pieces. In the case of doughs containing live yeasts, it is important to have yeasts capable of adapting to cold stress caused by the freezing and preservation at negative temperatures.

All the living cells naturally have systems for adapting to various stresses, which are designed to provide protection for the cell, its survival under stress conditions, and the repair of the damage caused by stress. These systems depend on a set of complex mechanisms which sometimes act separately, sometimes jointly, to allow adaptation to one or more types of stress.

Yeasts thus possess a set of complex mechanisms (for a review cf. ATTFIELD, Nature Biotech., 15, 1351-1357, 1997) which sometimes act separately, sometimes jointly, to allow adaptation to various types of stress.

Cold stress involves several components: an osmotic component and an ionic component, which are linked to the dehydration which occurs during freezing, and a component linked to the reduction in temperature, which slows the entire metabolism and in particular the transmembrane exchanges, and, through the formation of ice crystals, can damage the cellular structures.

The factors involved in cold stress tolerance in yeast are still poorly known. Most research studies carried out in order to enhance the tolerance of yeasts to stress, and in particular to cold stress, have targeted the increase in their trehalose content. It has thus been proposed to increase the synthesis of trehalose by overexpressing in yeasts strains genes encoding trehalose synthases (LONDESBOROUGH et al., U.S. Pat. No. 5,422,254), or alternatively to limit its catabolism by inactivating the ATH1 gene which encodes a trehalase (KLIONSKY et al., U.S. Pat. No. 5,587,290; KIM et al., Appl. Environ. Microbiol., 62, 1563-1569, 1996).

Numerous genes potentially involved in the resistance to stress have been identified in higher plants, and it has been proposed to use them to transform yeasts in order to increase their tolerance to stress.

Among these genes, there may be mentioned in particular the genes encoding proteins of the LEA (Late Embryogenesis Abundant) family which are highly expressed in plants during the maturation of the embryo. These genes are thought to play in particular a role in the tolerance of the embryo to desiccation.

Various groups of LEA have been distinguished, according to their structure, and according to their potential function (BRAY, Plant Physiology, 103, 1035-1040, 1993):

the group 1 LEAs are thought to prevent, through their high water-binding capacity, the loss thereof during desiccation;

the group 2 LEAs, also called dehydrins, are thought to play a role of chaperone proteins, contributing to maintaining the integrity of the structure of other proteins;

the group 3 and group 5 LEAs are thought to bring about sequestration of ions;

the group 4 LEAs are thought to have an osmoprotective role, and are thought to contribute to maintaining the integrity of the membranes.

IMAI et al. (Gene, 170, 243-248, 1996) report that the expression of the tomato LE25 protein (group 4 LEA) enhances the tolerance of S. cerevisiae to ionic stress and to cold stress (freezing). The same team ZHANG et al., (J. Biochem., 127, 611-616, 2000) also report that the expression in S. cerevisiae of tomato LE4 (group 2) and barley HVA1 (group 3) proteins also confers protection against cold stress, but to a lesser degree than LE25.

SWIRE-CLARK et al. (Plant Mol. Biol., 39, 117-128, 1999) have observed that the expression of the wheat Em protein (group 1 LEA) enhances the resistance of S. cerevisiae to osmotic stress, but has no effect on the resistance to cold stress.

The inventors have isolated and cloned various cDNAs corresponding to genes induced in hard wheat (Triticum durum) under abiotic stress conditions (ionic, osmotic, cold or thermal stress).

They studied the expression of these genes in S. cerevisiae and observed that some of them could enhance the tolerance of S. cerevisiae to various abiotic stresses. Among these, some are thought to provide in particular a very significant enhancement of tolerance to cold stress.

Thus, among the cDNAs tested, they selected:

a cDNA, called pTd38, encoding a group 2 LEA protein, described previously as being induced in hard wheat by desiccation (LABHILLI et al., Plant Sci., 112, 219-230, 1995); the nucleotide sequence of the cDNA pTd38 is represented in the sequence listing in the annex under the number SEQ ID NO: 1, the corresponding peptide sequence is represented under the number SEQ ID NO: 2;

a cDNA, called pTd64, encoding a WALI (for: “Wheat ALuminium Induced”) protein, which is very similar to the soft wheat (Triticum aestivum) WALI7 protein described by RICHARDS et al., (Plant Physiol., 105, 1455-1456, 1994; GenBank); the expression of the WALI genes was initially observed in the roots of soft wheat in response to a stress to aluminum. Their potential role in the response to other types of stress is not known. The nucleotide sequence of the hard wheat pTd64 cDNA is represented in the sequence listing in the annex under the number SEQ ID NO: 3, the corresponding peptide sequence is represented under the number SEQ ID NO: 4.

a cDNA, called pTd6.48, encoding a lipid transfer protein of the LTPs (for lipid transfer protein) of 9 kDa. The 9 kDa LTPs have the property of transferring in vitro discharged lipids between two membranes. Their role in vivo is still poorly known. It has been observed that their expression is induced under stress (abiotic or biotic) conditions; they inhibit the growth of various pathogenic microorganisms, and could thus play a role in the response to biotic stress (microbial attack). They could also be involved in the formation of the waxy cuticle of plants. The nucleotide sequence of the hard wheat pTd6.48 cDNA is represented in a sequence listing in the annex under the number SEQ ID NO: 5; the peptide sequences of the protein precursor, encoded by this cDNA, and of the mature LTP6.48 are represented under the numbers SEQ ID NO: 6 and SEQ ID NO: 7.

The subject of the present invention is the use of at least one nucleic acid molecule chosen from:

a) a nucleic acid molecule encoding a group 2 LEA protein whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 2;

b) a nucleic acid molecule encoding a WALI protein whose polypeptide sequence possesses at least 70% identity or 75% similarity, preferably at least 80% identity or 85% similarity, advantageously at least 90% identity or 9.5% similarity, and most preferably at least 95% identity or 98% similarity, with the sequence SEQ ID NO: 4;

c) a nucleic acid molecule encoding an LTP precursor or a mature LTP, whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 6 or the sequence SEQ ID NO: 7, respectively;

to transform a yeast in order to enhance its tolerance to cold stress.

The percentages of identity or of similarity between two polypeptide sequences may be determined according to methods known per se to persons skilled in the art; in particular, they may be determined with the aid of the BLASTP program (ALTSCHUL et al., Nucleic Acids Res., 25, 3389-3402, 1997).

These nucleic acid molecules may be used separately. They may also be combined in the same yeast cell, in order to further enhance its tolerance to cold stress, by combining various mechanisms participating in this tolerance; they may also be combined with nucleic acid molecules encoding proteins involved in mechanisms of tolerance to stress other than cold stress.

For example, to enhance the tolerance of a yeast cell to ionic stress, it is possible to express in this cell a nucleic acid molecule encoding a group 2 LEA whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 9. The inventors have indeed observed that the group 2 LEA, called Td27e, which is represented by the sequence SEQ ID NO: 9, has no effect on the tolerance of yeasts to cold stress, but enhances their tolerance to ionic stress.

Likewise, in order to enhance the tolerance of a yeast cell to osmotic stress, it is possible to express in said cell a nucleic acid molecule chosen from:

a nucleic acid molecule encoding a WALI protein whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 11;

a nucleic acid molecule encoding an LTP precursor or a mature LTP, whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 13 or the sequence SEQ ID NO: 14, respectively.

The inventors have indeed observed that the hard wheat WALI1 protein, which is represented by the sequence SEQ ID NO: 11, as well as the LTP called TdD2 which is represented in the sequence SEQ ID NO: 14, or its precursor, represented by the sequence SEQ ID NO: 13, have no effect on the tolerance of the yeasts to cold stress, but increase their tolerance to osmotic stress.

The subject of the present invention is also a yeast transformed with at least one nucleic acid molecule as defined above.

Transformed yeasts in accordance with the invention may be obtained from yeast strains of different species. By way of nonlimiting examples, there may be mentioned species of the genera Saccharomryces, Schizosaccharomyces, Kluyveromyces, Torulaspora, Pichia, Hansenula, Yarrowia, Candida, Geotrichum and the like.

Advantageously, said yeast belongs to a species which can be used in bread or pastry manufacture for the manufacture of raised doughs. It is in general a species of the genus Saccharomyces such as Saccharomyces cerevisiae.

Said yeast may thus be transformed with a nucleic acid molecule a) and/or a nucleic acid molecule b) and/or a nucleic acid molecule c) as defined above, optionally combined, as indicated above, with one or more other nucleic acid molecules which make it possible to confer on it resistance to stress other than cold stress.

Transformed yeasts in accordance with the invention may be obtained by the usual methods, known per se to persons skilled in the art. Conventionally, the DNA sequence encoding the protein which it is desired to express is inserted into an appropriate expression vector, under transcriptional control of a promoter which is active in a yeast cell. The vector which is thus charged is then introduced into the yeast cell, by any appropriate method, for example by electroporation or transformation with lithium acetate.

It is also possible to use extrachromosomal replicating vectors; by way of nonlimiting examples, there may be mentioned vectors of the Yep series (MYERS et al., Gene, 45, 299-310, 1986) which possess the replication origin of the endogenous 2μ plasmid, the Yrp vectors which possess, as replication origin, a chromosomal ARS sequence, and the like. It is also possible to use integrating vectors such as the Yip vectors, (MYERS et al., 1986) or Yiplac (GIETZ and SUGINO, Gene 74, 527-534, 1988) which do not possess a replication origin which is functional in yeast.

By way of example, there may be mentioned expression systems such as those described by BITTER et al. (Methods Enzymol., 153, 516-544, 1989) or TUITE (Expression of heterologous genes, 169-212, In M. F. TUITE and S. G. Oliver (ed.), Biotechnology Handbooks, Vol. 4, Saccharomyces., Plenum Press, New York, 1991).

The chosen gene may for example be placed under the control of elements for controlling regulation of transcription in yeasts such as those of the alcohol dehydrogenase ADH1 gene (RUOHONEN et al., J.

Biotechnol., 39, 193-203, 1995), the phosphoglycerate kinase PGK1 gene (HITZEMAN et al., Nucl. Acids Res., 10, 7791-7808, 1982), the glyceraldehyde-3-phosphate dehydrogenase TDH1 gene (BITTER and EGAN, Gene, 32, 263-274, 1984), the active ACT1 gene (GALLWITZ et al., Nucl. Acids Res., 8, 1043-1059, 1980), and the like.

The transformed yeasts in accordance with the invention exhibit a better tolerance to cold stress, which results in a survival level after freezing and storage at −20° C., greater than that of the wild-type strain from which they are derived. Advantageously, the rate of survival after freezing and storage at −20° C. is at least double that for the wild-type strain from which they are derived.

The transformed yeasts expressing the group 2 LEA as defined in a) above additionally exhibit a better tolerance to ionic stress and to heat stress than the wild-type strain from which they are derived; the transformed yeasts expressing a WALI7 protein as defined in b) above additionally exhibit a better tolerance to heat stress than the wild-type strain from which they are derived; the transformed yeasts expressing a 9 kDa LTP as defined in c) above additionally exhibit a better tolerance to ionic stress and to heat stress than the wild-type strain from which they are derived.

Transformed yeasts in accordance with the invention can be used in particular for the manufacture of baker's yeasts. The subject of the present invention is also baker's yeasts comprising said yeasts.

The present invention also encompasses bakery products, and in particular bakery doughs comprising said yeasts.

The subject of the present invention is in addition a lipid transfer protein chosen from:

an LTP precursor or a mature LTP whose polypeptide sequence possesses at least 90% identity of 95% similarity, and preferably at least 95% identity or 98% similarity with the sequence SEQ ID NO: 6 or the sequence SEQ ID NO: 7, respectively;

an LTP precursor or a mature LTP, whose polypeptide sequence possesses at least 70% identity or 75% similarity, preferably at least 80% identity or 85% similarity, advantageously at least 90% identity or 95% similarity, and most preferably at least 95% identity or 98% similarity, with the sequence SEQ ID NO: 13 or the sequence SEQ ID NO: 14, respectively.

The present invention also encompasses any nucleic acid sequence encoding one of said lipid transfer proteins.

A lipid transfer protein in accordance with the invention may, as indicated above, be expressed in yeasts in order to enhance their tolerance to cold shock. The present invention will be understood more clearly with the aid of the additional description which follows, which refers to examples describing the production of transformed yeast strains in accordance with the invention.

### EXAMPLE 1

### Construction of Expression Vectors and Selection of the Recombinant Yeasts

The cDNA encoding the Td38 group 2 LEA, the hard wheat WALI7 protein and the Td6.48 LTP were isolated from a cDNA library from the roots of hard wheat plantlets, which is obtained after induction by desiccation. The sequences of these cDNAs are respectively represented in the sequence listing in the annex under the numbers SEQ ID NO: 1, SEQ ID NO: 3 and SEQ ID NO: 5.

Vectors allowing the expression of these genes in S. cerevisiae were constructed from the E. coli/S. cerevisiae pYES2 shuttle expression vector (Invitrogen) possessing the ura3 gene, and the 2μ replication origin, by insertion under the transcriptional control of the gal1 promoter which is inducible by galactose and which is repressed by glucose, and the cyc1 transcription termination sequence.

Construction of a Group 2 LEA Expression Vector

The complete sequence of the pTd38 cDNA (SEQ ID NO: 1) encoding the Td38 protein (SEQ ID NO: 2) was inserted into the vector pYES2, digested beforehand with the enzymes NotI and KpnI and dephosphorylated.

The complete sequence of the cDNA, called pTd27e (SEQ ID NO: 8) encoding another group 2 LEA protein, called Td27e (SEQ ID NO: 9) whose expression in the roots of hard wheat plantlets is also induced by desiccation, and whose peptide sequence possesses 58.1% and 66.7% similarity with the sequence of the Td38 protein, was also cloned between the NotI and KpnI sites of pYES2.

Construction of a WALI Protein Expression Vector

The complete coding sequence of the pTdt64 cDNA (SEQ ID NO: 3) encoding a protein similar to the soft wheat WALI7 protein was inserted into the pYES2 vector, digested beforehand with the enzymes SacI and SphI and dephosphorylated.

The complete coding sequence of the cDNA called pTd79b (SEQ ID NO: 10), encoding a WALI1 protein, identical to the soft wheat WALI1 protein (SNOWDEN and GARDNER, Plant Physiol., 103, 855-861, 1993), and whose expression in the roots of hard wheat plantlets is also induced by desiccation, was cloned into pYES2 between the NotI and KpnI sites.

Construction of a 9 kDa LTP Expression Vector

The complete coding sequence of the pTd6.48 cDNA (SEQ ID NO: 5) encoding the LTP6.48 precursor (SEQ ID NO: 6) was inserted into the vector pYES2, digested beforehand with the enzymes EcoRI and SphI and dephosphorylated.

By way of comparison, a cDNA called pTdD2 (SEQ ID NO: 12), encoding the LTPD2 precursor (SEQ ID NO: 13), which exhibits 50% identity and 69% similarity with the LTP6.48 precursor, and whose expression in the roots of hard wheat plantlets is also induced by desiccation, was cloned into pYES2 between the NotI and KpnI sites.

The vectors thus obtained were used to transform the Saccharomyces cerevisiae INVSc1 strain (haploid strain having the genotype MATα, his3-Δ1, leu2, trp1-289, ura3-52). This strain is cultured on a YNB-Gal medium composed of 1.7 g of YNB-AA/AS (DIFCO LABORATORIES), 5 g of (NH₄)₂SO₄, 20 g of galactose, 20 mg of uracil, 20 mg of L-histidine, 60 mg of L-leucine, 40 mg of L-tryptophan for 1 liter. For the selection of the recombinant yeasts, the same medium is used without uracil. After selection of the yeasts, the presence of the transcripts corresponding to the cDNAs is verified by hybridization with specific probes after induction with galactose.

### EXAMPLE 2

### Stress tolerance of the Recombinant Yeasts

The tolerance to cold stress was studied in the wild-type strain INVSc1, in the INVSc1 strain transformed with the empty plasmid pYES, and in the recombinant strains expressing a type 2 LEA, a WALI protein or a 9 kDa LTP obtained as described in example 1 above.

Cold Stress:

Stress Conditions

The tolerance to cold stress is evaluated by measuring the survival of the yeasts after application of the cold stress (freezing at −20° C., preservation at this temperature for 24 hours, and thawing). The conditions described by IMAI et al. (1996, publication cited above) were used.

Each of the strains is cultured on YNB-Galactose medium (50 ml). In the middle of the exponential growth phase (Absorbance at 600 nm=1), 1 ml of culture is removed. After centrifugation (3 500 g, 3 min, 4° C.), the cellular pellet is resuspended in 1 ml of complete YPD medium. 100 μl aliquots are separated into microtubes. A portion of the aliquots is stored without treatment, and the other is frozen in a bath of absolute ethanol cooled beforehand and stored at −20° C. for 24 h. The cells are rapidly thawed by placing the tubes at 30° C. for a few seconds before spreading.

The untreated cells or the thawed cells are then diluted in complete YPD medium (1/10 000 and 1/20 000 dilution for the controls, and 1/400 and 1/20 00 for the cells which have been subjected to cold stress). The dilutions thus obtained are spread on complete agar medium (YPD agar) and the dishes incubated at 30° C. for 48 h. The colonies formed on each dish are counted, and the number of CFU (colony forming units) is calculated taking account of the dilution.

The survival of each strain is evaluated by the percentage of surviving CFUs, that is to say the number of CFUs after stress divided by the number of control CFUs. For each experiment, the number of CFUs corresponds to the mean of 4 spreadings.

The results are summarized in table I below

TABLE I

| Strain | Survival rate |
|---|---|
| pYES2 | 6.8 |
| wild-type yeast | 5.5 |
| pYES2-Td27e | 6 |
| pYES2-Td38 | 20 |
| pYES2-Td79b | 3.5 |
| pYES2-Tdt64 | 27 |
| pYES2-ltp6.48 | 28 |
| pYES2-ltpD2 | 1.8 |

In the case of the wild-type strain INVSc1, the survival rate is 5.5%; the recombinant strain INVSc1-pYES2 exhibits a similar survival rate of 6.8%.

Recombinant Yeasts Expressing a Group 2 LEA Protein

The recombinant yeast INVSc1-pYES2-Td27e has a 6% rate of survival to cold stress, which is therefore similar to those observed with the wild-type strain INVSc1 or the strain INVSc1-pYES2.

On the other hand, the recombinant strain INVSc1-pYES2-Td38 has a 20% rate of survival to cold stress.

Recombinant Yeasts Expressing a WALI Protein

The recombinant yeast INVSc1-pYES2-Td79b has a 3.5% rate of survival to cold stress, comparable to those for the wild-type strain INVSc1 or the strain INVSc1-pYES2.

On the other hand, the recombinant yeast INVSc1-pYES2-Tdt64 has a 27% rate of survival to cold stress.

Recombinant Yeasts Expressing an LTP

The recombinant yeast INVSc1-pYES2-ltpD2 has a 1.8% rate of survival to cold stress. The expression of this LTP therefore induces no enhancement of the survival to cold stress.

The recombinant yeast INVSc1-pYES2-ltp6.48 has, on the other hand, a 28% rate of survival to cold stress.

Heat Stress:

Stress Conditions

The tolerance to heat stress is evaluated according to the same protocol as for cold stress: instead of a stress by freezing, the cells are subjected to incubation for 10 minutes at 50° C. The cells subjected to heat stress are diluted to ¼ 000 and 1/10 000 before spreading. The survival rate is calculated as described above for cold stress.

The results are illustrated in Table II below.

TABLE II

| Strain | Survival rate |
|---|---|
| pYES2 | 24 |
| wild-type yeast | 27.5 |
| pYES2-Td27e | 77.5 |
| pYES2-Td38 | 91 |
| pYES2-Td79b | 46 |
| pYES2-Tdt64 | 56 |
| pYES2-ltp6.48 | 100 |
| pYES2-ltpD2 | 34.5 |

In the case of a wild-type strain INVSc1, the survival rate is 27.5%; it is 24% for the recombinant strain INVSc1-pYES2.

All the expressed proteins induce an increase in the survival rate. This increase is particularly high in the case of Td38 and of LTP6.48. It is also high in the case of Td27e.

Ionic Stress and Osmotic Stress:

Stress Conditions

The tolerance to ionic stress or to osmotic stress is evaluated by comparing the growth kinetics at 30° C. under normal conditions (YNB-Galactose medium), and under ionic stress conditions (YNB-Galactose+1.5 M Nacl medium), or under osmotic stress conditions (YNB-Galactose

+2.5 M sorbitol medium).

In the case of the wild-type strain INVSc1, or of the recombinant strain INVSc1-pYES2, the duration of the log phase (defined here as the period between the initiation of the culture and the time when the absorbers at 600 nm reaches a value of 0.2), is 3 h under normal culture conditions. In the presence of 1.5 M NaCl, the duration of the log phase is 87 h. In the presence of 2.5 M sorbitol, the log phase is 182 h.

Recombinant Yeasts Expressing a Group 2 LEA Protein

INVSc1-pYES2-Td27e

In the presence of 1.5 M NaCl, the log phase of the growth kinetics for the yeast INVSc1-pYES2-Td27e is 70 h less than that for the control yeasts (wild-type strain INVSc1, or recombinant strain INVSc1-pYES2) under the same conditions.

On the other hand, in the presence of 2.5 M sorbitol, the log phase for the transformed yeast is 48 h more than that for the control yeasts.

INVSc1-pYES2-Td38

In the presence of 1.5 M NaCl, the log phase of the growth kinetics for the yeast INVSc1-pYES2-Td38 is 44 h less than that for the control yeasts.

In the presence of 2.5 M sorbitol, no reduction in the growth phase is observed compared with the control yeasts.

It therefore appears that the yeast INVSc1-pYES2-Td27e, and to a lesser degree the yeast INVSc1-pYES2-Td38, have an enhanced tolerance to an ionic stress.

On the other hand, none of the transformed yeasts have an enhanced tolerance to an osmotic stress. The yeast INVSc1-pYES2-Td27e appears even more sensitive than the control yeasts.

Recombinant Yeasts Expressing a WALI Protein

INVSc1-pYES2-Tdt64

Under osmotic stress conditions, the growth kinetics for the yeast INVSc1-pYES2-Tdt64 is not modified. Only a slight decrease (10 h) in the log phase is observed in the presence of 1.5 M NaCl.

INVSc1-pYES2-Td79b

Compared with the control yeasts, the log phase for the transformed yeast INVSc1-pYES2-Td79b is decreased by 10 h in the YNB-Galactose medium supplemented with 1.5 M NaCl and by 107 h in the YNB-Galactose medium supplemented with 2.5 M sorbitol.

It therefore appears that the expression of the Td79b (WALI1) protein confers an enhanced tolerance to an osmotic stress, and a slight increase in the tolerance to an ionic stress. The expression of the (WALI7) protein only confers a slight increase in the tolerance to an ionic stress.

Recombinant Yeasts Expressing an LTP

INVSc1-pYES2-ltpD2

Compared with that for the control yeasts, the log phase for the yeast INVSc1-pYES2-ltpD2 is shortened by 50 h in the YNB-Galactose medium supplemented with 1.5 M NaCl, and by 25 h in the presence of 2.5 M sorbitol.

INVSc1-pYES2-ltp6.48

The log phase for the yeast INVSc1-pYES2-ltp6.48 is shortened by 50 h, compared with that for the control yeasts, in the presence of 1.5 M NaCl. It does not differ from those for the control yeasts in the presence of 2.5 M sorbitol.

It therefore appears that the tolerance to ionic stress is enhanced by the expression of the LTPD2 protein or of the LTP6.48 protein. On the other hand, only the expression of LTPD2 confers an enhanced tolerance to an osmotic stress.

### EXAMPLE 3

Cold Stress Tolerance of Recombinant Yeasts Obtained From an Industrial Strain

Host Yeast Strain:

Industrial strain used in bread making=Saccharomyces cerevisiae CLIB 320 (cellobiose−, D-galactose+, D-glucose+, lactose−, maltose+, melezitose+, melibiose−, raffinose+, saccharose+, trehalose−).

CLIB=collection de souches d'intérêt biotechnologique de Grignon [Collection of strains of biotechnological interest from Grignon].

Expression Vector: PVT100-U-ZEO

PVT100-U-ZEO is derived from the vector PVTU (VERNET et al., Gene, 52, 2325-2333, 1987). The characteristics of this vector are the following:

shuttle vector with the replication origin 2μ for the yeast and ori for E. coli;

ampicillin resistance (AmpR) for selection in E. coli and phleomycin resistance (ZEO) for selection in the yeast;

Adh promoter and Adh terminator.

Construction of the Expression Vectors and Selection of Their Recombinant Yeasts:

The vector PVT100-U-ZEO is digested with the enzymes XbaI and XhoI and dephosphorylated. The same XbaI and XhoI sites are created, by PCR, at the ends of the sequences encoding the group 2 LEA Td38 (SEQ ID NO: 2), the hard wheat WALI7 protein Tdt64 (SEQ ID NO: 4), the LTP Td6.48 (SEQ ID NO: 6), and the LTP Td6.48 without the signal peptide (SEQ ID NO: 7), in order to allow oriented cloning of these cDNAs. The constructs obtained were verified by sequencing.

Each of the recombinant vectors and the empty vector are used for the transformation of the CLIB320 strain. The lithium acetate method of transformation described by GRISHIN and KORSHUNOVA (Yeast Genetics and Molecular Biology, The Hague, 1990) was used.

The yeasts are cultured on YEPD medium (10 g of yeast extract, 20 g of peptone (DIFCO Laboratories), 20 g of D(+)−glucose for 1 l). An agar medium is obtained by adding 20 g of bacto-agar to the preceding composition. The transformants are selected on the basis of the resistance to phleomycin. For that, 100 μg/ml of phleomycin (CAYLA) are added to the YEPD medium.

Stress Tolerance of the Recombinant Yeasts:

Stress Conditions:

The tolerance to cold stress of each yeast strain is evaluated by the rate of survival after application of the stress. Two types of stress were applied: rapid freezing at −20° C. and preservation at −20° C. for variable periods of time, and then thawing at room temperature or thawing by placing at 4° C. for 24 h.

Experiments:

A preculture of each recombinant strain and the wild-type strain is carried out by inoculation, with an isolated colony, of 50 ml of YEPD medium+100 μg/ml of phleomycin and culturing at 28° C., 220 rpm, 48 h. 100 ml of YEPD medium+100 μg/ml of phleomycin are inoculated with 1/20th of the volume of the preculture and cultured under the same conditions until an absorbance at 660 nm of 4 is reached, plateau phase of the growth kinetics. Platings after dilution are carried out at the time 0 on YEPD medium supplemented with 100 μg/ml of phleomycin, and then incubated in an incubator at 30° C. for 48 h. Aliquot portions of 500 pl of culture are frozen at −20° C. by placing them in a container of cold absolute ethanol. Every 24 h, an aliquot portion of each sample is thawed at room temperature and another is placed at 4° C. for 24 h for a gentle thawing. Platings at serial dilutions are carried out and then incubated in an incubator at 30° C. for 48 h. For each thawing period, the number of colony forming units (cfu) relative to the number of cfu developing at time 0 allows the survival rate to be determined (expressed as %).

For each duration of freezing and each stress condition, the number of cfu corresponds to the mean of the results obtained for two clones and for two platings per clone. The results expressed by the survival rate (%) are summarized in the following tables III and IV.

TABLE III
Stress −20° C. and then thawing at 4° C.

| | | Yeast CLIB 320 transformed with: | | | | |
|---|---|---|---|---|---|---|
| | | | | | | PVT100-U- |
| | | | | | | ZEO |
| Duration | | | | | | Td6.48 |
| of | Wild-type | | | | PVT100-U- | without |
| freezing | yeast | PVT100-U- | PVT100-U- | PVT100-U- | ZEO | signal |
| in days | CLIB 320 | ZEO | ZEO Tdt64 | ZEO Td38 | Td6.48 | peptide |
| 0 | 100 | 100 | 100 | 100 | 100 | 100 |
| 1 | 32 | 28.5 | 45.5 | 53.5 | 54.5 | 57 |
| 2 | 29.5 | 23.5 | 52.5 | 20.5 | 26 | 35 |
| 3 | 20 | 18 | 34.5 | 23 | 24 | 30 |
| 5 | 3 | 8.5 | 13 | 27 | 10 | 13.5 |
| 8 | 0.5 | 1.8 | 4.5 | 11.5 | 4.5 | 2.5 |

TABLE IV
Stress −20° C. and then thawing at room
temperature

| | | Yeast CLIB 320 transformed with: | | | | |
|---|---|---|---|---|---|---|
| | | | | | | PVT100-U- |
| | | | | | | ZEO |
| Duration | | | | | | Td6.48 |
| of | Wild-type | | | | PVT100-U- | without |
| freezing | yeast | PVT100-U- | PVT100-U- | PVT100-U- | ZEO | signal |
| in days | CLIB 320 | ZEO | ZEO Tdt64 | ZEO Td38 | Td6.48 | peptide |
| 0 | 100 | 100 | 100 | 100 | 100 | 100 |
| 1 | 33.5 | 29 | 28.5 | 38.5 | 44.5 | 35 |
| 2 | 29.5 | 11 | 23.5 | 32 | 18 | 16.5 |
| 3 | 13 | 5.5 | 15 | 25 | 16.5 | 10 |
| 5 | 6 | 2 | 11.5 | 23 | 7.5 | 8.5 |
| 8 | 3 | 2.5 | 5.5 | 10 | 3.5 | 3 |

1. The use of at least one nucleic acid molecule chosen from:
a) a nucleic acid molecule encoding an LTP precursor or a mature LTP whose polypeptide sequence possesses at least 70% identity or 75% similarity with the sequence SEQ ID NO: 6 or the sequence SEQ ID NO: 7 respectively;
b) a nucleic acid molecule encoding a WALI protein whose polypeptide sequence possesses at least 70% identity or 75% similarity with the sequence SEQ ID NO: 4;
c) a nucleic acid molecule encoding a group 2 LEA protein whose polypeptide sequence possesses at least 70% identity or 75% similarity with the sequence SEQ ID NO: 2;
to transform a yeast in order to enhance its tolerance to cold stress.

2. A yeast transformed with at least one nucleic acid molecule as defined in claim 1.

3. The transformed yeast as claimed in claim 2, characterized in that it is additionally transformed with at least one nucleic acid molecule chosen from:
a nucleic acid molecule encoding a group 2 LEA protein whose polypeptide sequence possesses at least 70% identity or 75% similarity with the sequence SEQ ID NO: 9;
a nucleic acid molecule encoding a WALI protein whose polypeptide sequence possesses at least 70% identity or 75% similarity with the sequence SEQ ID NO: 11;
a nucleic acid molecule encoding an LTP precursor or a mature LTP whose polypeptide sequence possesses at least 70% identity or 75% similarity with the sequence SEQ ID NO: 13 or the sequence SEQ ID NO: 14 respectively.

4. The transformed yeast as claimed in either of claims 2 and 3, characterized in that said yeast belongs to the genus Saccharomyces.

5. The transformed yeast as claimed in claim 4, characterized in that said yeast belongs to the species Saccharomyces cerevisiae.

6. A baker's yeast, comprising a transformed yeast as claimed in any one of claims 2 to 5.

7. A bakery product comprising transformed yeasts as claimed in any one of claims 2 to 5.


SEQUENCE LISTING (14 sequences)

SEQ ID NO: 1 - DNA, 555 bp, Triticum durum
acacaaccaagacaagtaaacagcagcactagtagatttcccgagtgacaagttcagcgcaacatggagcaccagggacacggcaccggcgagaagaagggcatcatggagaacatcaaggagaagctccccggtggccaaggtgaccaccagcagaccgctggcacccacgggcagcatggacacactggaatgacaggcacggagatgcatgacaccacggccaccggcggcacccatgggcagcaggggcttaccggaacgactggcactgggacacacggcaccggtgagaagaagagcctcatggacaaggtgaaggagaagctgcctggacagcactaagctcggtctgcccacggccgccacctttgcagaataatactccaccgtatatgaattgatctgagtctagttcacctagctcacttggtcgttggaggagcaaatgtatctctggtttaagttttcacggacaacagtgtgttcacagttttcgtctatttacactccgtcatgcaaatttcctttttgttccaaaaaaaaaaaaaaaaa

SEQ ID NO: 2 - protein, 93 aa, Triticum durum
MEHQGHGTGEKKGIMENIKEKLPGGQGDHQQTAGTHGQHGHTGMTGTEMHDTTATGGTHGQQGLTGTTGTGTHGTGEKKSLMDKVKEKLPGQH

SEQ ID NO: 3 - DNA, 891 bp, Triticum durum
agtgaggaaggccacaatcagcaactgacctgtaataccttacctagctaggtgtactatgtttgagccaagatgttgggggtgttcagcggcgaggtggtggaggtgccggcggagctggtggcggccgggagccggacgccatcgcccaagacacgggcgtcggagctggtgaagcgcttcctcgccggcaacgacctggccgtgtccgtggagctgggatcactgggcaacctcgcctactcccacgccaaccagtccctcctcctcccaaggtctttcgctgcaaaggatgagatcttctgcctgttcgagggagtcctggacaacttggggcggttgagccagcagtacggcctctccaagggcggcaacgaggtgctcctcgtgatcgaggcctacaagacgctgagggacagagccccctatcccgccagcttcatgctctcccagctcaccggcagctacgccttcgtgctcttcgacaagtccacctcctccctcctcgtcgcatccgacccggagggcaaggtgccgctcttctggggaatcaccgccgacggctgcgtcgccttctccgacgacatcgacctgctgaaaggatcttgcggcaagtcactggcgcctttcccgcaaggttgcttctactggaacgctcttggaggcctcaagtcgtacgagaatcccaagaacaaggtcaccgctgtgcctgcagatgaggaggaaatctgtggtgcaactttcatggtggaaggatctaccgttgtcgcggcacttcagtaggagattctcttgtctccgtcgctgggcaaagcaggcaaggccgttcgtgtgtagatggtggtggtgtaatataatgcaacaaggcgcgtgtgctactctcttgtgatc

SEQ ID NO: 4 - protein, 236 aa, Triticum durum
MLGVFSGEVVEVPAELVAAGSRTPSPKTRASELVKRFLAGNDLAVSVELGSLGNLAYSHANQSLLLPRSFAAKDEIFCLFEGVLDNLGRLSQQYGLSKGGNEVLLVIEAYKTLRDRAPYPASFMLSQLTGSYAFVLFDKSTSSLLVASDPEGKVPLFWGITADGCVAFSDDIDLLKGSCGKSLAPFPQGCFYWNALGGLKSYENPKNKVTAVPADEEEICGATFMVEGSTVVAALQ

SEQ ID NO: 5 - DNA, 782 bp, Triticum durum
aatacgactcactatagggaaagctggtacgcctgcaggtaccggtccggaattcccgggtcgacccacgcgtccggaaaatctagctatctcatcatctctgcctgagctcactaccactactattgctagcttgatcgagatggcccgttctgctgttgctcaggtcgtgctcgtcgccgtggtggctgctatgctcctcgcagtcacggaggcggctgtatcgtgcggtcaggtgagctctgccttgagcccctgcatctcctatgcacgcggcaacggcgccagcccatctgcggcctgctgcagcggcgttaggagtctagccagctcagcccggagcaccgctgacaagcaagcggcgtgcaagtgcatcaagagcgctgctgctgggctcaacgctggcaaggccgccggcatccccacaaagtgcggcgttagcgtcccttacgccatcagctcttcggtcgactgctctaagattcgctgatcgagcacttgctgccatcgctgttgccatcgtcccctacgccatcgttgctggatctacgcttagtacgttgaggtcacacacacgcacacccacatatatatatgaataaatgctctcatattatctcactgcgtgagagagagaggagtacgtacgtccaagcagctctgcatggccggccacactgttgtatcgatgtttggttgttcttccactccccgagtttgctgtactttgtaccatgtgtacttttgatatatggattgtgtactcagctgatcagctctaa

SEQ ID NO: 6 - protein, 116 aa, Triticum durum
MARSAVAQVVLVAVVAAMLLAVTEAAAVSCGQVSSALSPCISYARGNGASPSAACCSGVRSLASSARSTADKQAACKCIKSAAAGLNAGKAAGIPTKCGVSVPYAISSSVDCSKIR

SEQ ID NO: 7 - protein, 90 aa, Triticum durum
AVSCGQVSSALSPCISYARGNGASPSAACCSGVRSLASSARSTADKQAACKCIKSAAAGLNAGKAAGIPTKCGVSVPYAISSSVDCSKIR

SEQ ID NO: 8 - DNA, 751 bp, Triticum durum
caaagagcaaaagctaaagccacaaccaagtccagtttaggaagaggcagagatggagttccaagggcagcacgacaaccccgccaaccgcgtcgacgagtacggcaacccgttcccgctggccggcggcgtggggggaggacacgccgctcctggcaccggcgggcagttacaggcccgcaggggagagcacaagaccggtgggatcctgcatcgctccggcagctccagctccagctcgtcttccgaggacgacggcatgggcgggaggaggaagaagggcatgaaagagaagatcaaggagaagctccccggcggccacaaggacaaccagcagcacatggcgactggtacagggactggaggagcctacgggccggggactggaactggtggagcctacgggcagcaaggccacgcaggaatggccggcgccggcactggcaccggcgagaagaaggggatcatggacaagattaaggagaagctgccgggacagcactgagccgacggctccggctggccgcttcctttgcatagctacacgcgtcaatgccttctagttccacgtgatctttttgttcaataataataagatgaagcagaacgaaaacttgtctctgatctcgtctgtgtcagggacacttttctgtatacagcgtgcgtcgtgtttgttatgttttgtgtgttgtgtcttcatgttgaaacaaatttagtgtacaactgaaaaaaaaaaaaaaaaaaaaaaaaa

SEQ ID NO: 9 - protein, 150 aa, Triticum durum
MEFQGQHDNPANRVDEYGNPFPLAGGVGGGHAAPGTGGQLQARRGEHKTGGILHRSGSSSSSSSSEDDGMGGRRKKGMKEKIKEKLPGGHKDNQQHMATGTGTGGAYGPGTGTGGAYGQQGHAGMAGAGTGTGEKKGIMDKIKEKLPGQH

SEQ ID NO: 10 - DNA, 551 bp, Triticum durum
catcatcctcgacaccaaagctcatcttcttctccttgaaatctttttgggttcatcagatttggaggatgtcttgcaactgtggatccggttgcagctgcggctcagactgcaagtgcgggaagatgtaccctgatctgacggagcagggcagtgccgcggcccaggtcgccgccgtggtcgtcctcggcgtggcgcctgagaacaaggcggggcagttcgaggtggccgccggccagtccggcgagggctgcagctgcggcgacaactgcaagtgcaacccctgcaactgttaagctgcatgcactcgtgtgatggtgtgagagtatacgtgaataacgagcgtccctctgatctgatggagtcgagcaagggtgcgtgtgcgtgtgcgtgtggtttacttgctcgctctccgcctatgctctgcccttggtgtccttgtgtgtatgtgtgtgcacgtgtccctgtaattgcttcatctatctccactatggatggagtgatgaatatgtaagaatgaatgatttacctaaaaaaaaaaaaaaaaaaaa

SEQ ID NO: 11 - protein, 75 aa, Triticum durum
MSCNCGSGCSCGSDCKCGKMYPDLTEQGSAAAQVAAVVVLGVAPENKAGQFEVAAGQSGEGCSCGDNCKCNPCNC

SEQ ID NO: 12 - DNA, 591 bp, Triticum durum
acatttccagcaagcaagccgaagcactagatcctcgatggctcgcgtggcactgctcgccgtgttcaccgtgctcgccgcactggcagtggcggagatggcgtctggggcggtgacctgcagcgacgtgacgtccgccatcgcgccgtgcatgtcctacgcaacggggcaagcgtcgtcaccctcggcggggtgctgcagcggggtgaggaccctgaacggcaaggcgtccacctcggccgaccggcaggcggcgtgccgctgcctcaagaacctggcggggtcgttcaatggcatcagcatgggtaacgccgccaacatccccggcaagtgcggcgtctccgtctctttccccatcaacaacagcgtcaactgcaacaaccttcattaagttatctacgagcatcatcatcacaccaggctagctagcccactcggtgtctactgttgctgctctctgcgtgtgttcgttgttgttttctgcatgtgttccacctccatctgttgtccttgttacagatcgagcagattactgatcgaatcatcaataaaataatgtgttgagcggaagtttttaaaaaaaaaaaaaaa

SEQ ID NO: 13 - protein, 117 aa, Triticum durum
MARVALLAVFTVLAALAVAEMASGAVTCSDVTSAIAPCMSYATGQASSPSAGCCSGVRTLNGKASTSADRQAACRCLKNLAGSFNGISMGNAANIPGKCGVSVSFPINNSVNCNNLH

SEQ ID NO: 14 - protein, 93 aa, Triticum durum
AVTCSDVTSAIAPCMSYATGQASSPSAGCCSGVRTLNGKASTSADRQAACRCLKNLAGSFNGISMGNAANIPGKCGVSVSFPINNSVNCNNLH